(19) United States
(12) Patent Application Publication
(10) Pub. No.: US 2007/0005006 A1
(43) Pub. Date: Jan. 4, 2007

(54) Methods and devices for removal of toxic compounds from breast milk

(76) Inventor: Paul Edward Rosenfeld, Venice, CA (US)

Correspondence Address: GREENBERG TRAURIG LLP, 2450 COLORADO AVENUE, SUITE 400E, SANTA MONICA, CA, 90404, US

(21) Appl. No.: 11/153,818

(22) Filed: Jun. 15, 2005

(51) Int. Cl. A61M 1/06

(52) U.S. Cl. 604/73; 215/11.1

(57) ABSTRACT

Filter devices are described for removing organic toxins and/or inorganic toxins, such as halogenated endocrine disruptors, heavy metals and radionuclides from breast milk. The filter device can comprise a nipple shield device having a filter positioned in the interior of the nipple shield. The nipple shield device can be positioned on a female mammalian breast to filter expressed breast milk. Alternatively, the baby bottle can incorporate a filter device. Further, a breast milk pump can comprise a filter that is positioned interior to a breast milk pump to filter the breast milk as it is collected. General methods are described for removal of toxins from breast milk through filtration.

### FIELD OF THE INVENTION

The invention relates to methods and devices for removing toxic compounds, such as halogenated endocrine disruptors, phthalates, heavy metals, and radionuclides from breast milk. In particular, the invention relates to a filter device that can be used, for example, in conjunction with a nipple shield, a bottle or a breast pump, to remove toxic compounds from breast milk.

### BACKGROUND OF THE INVENTION

The benefits of breastfeeding an infant are well recognized, providing nutritional, physiologic and psychological benefits to the breastfeeding child. Breastfeeding may provide benefits such as lower rates of asthma, diabetes, some childhood cancers, pneumonia, diarrhea, and ear infections, to the breastfed child. In addition, it appears that breastfeeding may reverse some of the damage that may be caused to the fetus by exposure to chemicals while in the womb.

Breastfeeding can be accomplished through direct feeding of the infant or by collecting the breast milk for subsequent consumption. The direct feeding of the infant can involve a nipple shield that provides relief for the nursing mother from potential soreness or injury from the child. A breast shield can also help the baby nurse. Milk can also be collected from the mother using a breast pump that uses a manual or electric pump to collect the milk. The collected breast milk can be put into a baby bottle with a nipple on its end for later feeding to the baby. A range of breast pumps is commercially available.

### SUMMARY OF THE INVENTION

In a first aspect, the invention pertains to a filtering nipple shield comprising a nipple structure and a first filter. The nipple structure comprises a nipple shaped material having an interior and a tip with one or more holes. The first filter comprises a first filtration medium. The first filter can be positioned in the interior of the nipple structure such that the filtration medium effectively removes organic or inorganic toxins from liquids contacting the filtration medium.

In a further aspect, the invention pertains to a baby bottle comprising a container, a nipple and a filter. The container comprises an opening and an attachment portion. The nipple comprises a lip with a flange, a protrusion and a tip with one or more holes. The nipple fits over the opening of the container. The filter comprises a filtration medium that effectively removes organic or inorganic toxins from liquids contacting the filtration medium. Generally, the filter is positioned within the bottle between the container and the tip of the nipple.

In another aspect, the invention pertains to a filtering breast pump comprising a reservoir, a breast cup, a filter and a pump. The breast cup comprises a shaped cone and a neck extending from the cone fluidly connected to the reservoir such that breast milk generated in the breast cup flows through the neck into the reservoir. The filter is positioned within the flow path between the breast cup and the reservoir. The pump is positioned to evacuate the reservoir to induce pumping through the neck to the breast cup.

In an additional aspect, the invention pertains to a method for removing organic or inorganic toxins from breast milk. The method comprises filtering breast milk with a filter effective to remove organic toxins or inorganic toxins.

### BRIEF DESCRIPTION OF THE DRAWINGS

FIG. 1 is a perspective view of a nipple shield filter device showing a position of the filter in the nipple shield filter.

FIG. 2 is a perspective view of a nipple shield filter device showing an alternative positioning of the filter within the nipple filter device.

FIG. 3 is a cross-sectional view of a nipple shield filter device showing one configuration of the filter within the nipple filter device.

FIG. 4 is a perspective view of a nipple shield filter device showing a plurality of filters positioned in the nipple shield filter device.

FIG. 5 is an exploded perspective view of a filtering baby bottle.

FIG. 6 is a perspective view of a nipple filter device showing an alternate positioning of the filter in the nipple filter device.

FIG. 7 is a perspective view of a nipple filter device showing an alternate configuration of the filter in the nipple filter device.

FIG. 8 is a sectional schematic view of a breast milk pump showing a positioning of the breast milk pump filter within the breast milk pump.

### DETAILED DESCRIPTION OF THE INVENTION

Breast milk delivery devices are described that provide for the removal of toxic or undesirable compounds from the breast milk prior to provision of the milk to the nursing child. The compounds can be removed through filtration without significantly interfering with the nursing process. Suitable filters are selected to remove undesirable organic and/or inorganic compounds from the breast milk. The filters can be associated with a nipple shield, a baby bottle and/or a breast milk pump to remove the toxins at feeding or at collection of the milk.

Filter devices have been designed to remove toxins, such as halogenated endocrine disruptors, phthalates, heavy metals and radionuclides, from breast milk. In some embodiments, the filter device comprises a nipple shield having a base and a protrusion that is shaped to conform to a mammalian female areola and nipple. The protrusion comprises one or more holes, thus allowing breast milk to pass through the holes and permitting intake of breast milk by an infant. The nipple shield has a filter material, such as activated carbon and/or a resin that sorbs (i.e., absorbs or adsorbs) cations and anions, organic and inorganic toxins, and endocrine disruptors. The filter material is along the flow path from the interior of the nipple shield to the holes in the nipple tip, such that the milk from the nipple is filtered prior to ingestion by the suckling infant. The nipple shield can be adapted from a type of nipple shield used to protect cracked and otherwise sore female mammalian nipples.

In another embodiment, the filter device can be used in association with a baby bottle. The filter bottle comprises a nipple having a base, a protrusion and filter media positioned within the nipple. The protrusion comprises at least one hole, through which the infant can obtain the milk contained in the baby bottle. The nipple comprises a filtration material, such as activated carbon and/or resins that sorb cations and anions, organic and inorganic toxins, and endocrine disruptors. The bottle portion of the filter bottle can be adapted from a range of designs, such as presently available commercial designs.

In other embodiments, a breast milk pump can be adapted to comprise a filtration medium for the removal of toxins, such that breast milk passes through the filtration medium prior to collection in a receptacle. A filter device can comprise, for example a filter packet, wherein the packet further comprises, for example, activated carbon and/or resins that sorb cations and anions, organic and inorganic toxins, and endocrine disruptors. A filter packet can take on various shapes depending upon the design and needs of a particular breast milk pump, while enabling convenient replacement of the packet and cleaning of the pump.

The filter devices described herein reduce or eliminate the amount of halogenated endocrine disruptors, phthalates, heavy metals and radionuclides passing via breast milk to the feeding infant by filtering the halogenated endocrine disruptors, phthalates, heavy metals, and radionuclides out of the breast milk prior to ingestion by the infant. The filter device can be used when directly breast feeding an infant, bottle-feeding an infant or when pumping breast milk for storage and later use in feeding an infant.

As more chemicals from, for example, farming and industrial activity enter the environment and find their way into foodstuffs and drinking water, some of these chemicals can appear in breast milk. The appearance of these chemicals in breast milk is causing concern due to the ingestion of these chemicals by infants. Breast milk is comprised from fat from the mother's body and may contain chemicals that have accumulated in the mother's body fat, such as polychlorinated biphenyls (PCBs) and polybrominated diphenyl ethers (PBDEs). Breast milk has been found to contain halogenated endocrine disruptors, such as, PCBs and PDBEs as well as dioxins, polychlorinated dibenzofurans, perchlorates and other endocrine disruptors.

Research on environmentally related chemical contaminates in breast milk has been conducted, with special concern in those areas of the world where breast milk may be the only source of food for the infant. Varying levels of organochlorine pesticides, polychlorinated biphenyls (PCBs), polychlorinated dibenzodioxins (PCDDs), polychlorinated dibenzofurans (PCDFs), polybrominated diphenyl ethers (PBDEs), heavy metals and solvents have been found in breast milk. Levels of the organochlorine pesticides, PCBs, PCDFs, PBDEs and dioxins have declined in breast milk in countries where these chemicals have been banned or otherwise regulated.

Generally, however, the levels of PBDEs are rising due to the increased use of PBDE's as a flame-retardant in an increasing number of products, including household products. Polybrominated diphenyl ethers (PBDEs) are a group of flame-retardants that have been added to upholstered furniture and consumer electronics since the mid 1970s. PBDEs have been detected in the environment since the 1980s and have begun appearing in breast milk. These PBDEs may disrupt thyroid hormones levels during pregnancy and may have a negative effect on brain development in the developing fetus. PBDE along with PCB and other endocrine disruptors affect the thyroid hormones in the third trimester of pregnancy when brain connections are forming in the developing fetus. These endocrine disruptors may create brain deficits and make it more difficult for children exposed to endocrine disruptors to learn.

Inorganic toxins for removal include, for example, heavy metals and/or radionuclides. The term heavy metal refers to metallic chemical elements or corresponding ions that have high molecular weights. Heavy metals include, for example, cadmium, arsenic, lead, mercury, nickel, chromium, copper, selenium and zinc. Heavy metals generally are toxic in low concentrations and may bioaccumulate. However, trace amounts of some heavy metals, such as copper and zinc are required by living organisms.

Some toxic heavy metals, such as lead, cadmium, mercury and arsenic have been found in breast milk The heavy metals that are found in breast milk are transferred to the infant and may bioaccumulate in the infant's body. The heavy metals can have various deleterious effects on an infant, such as points lost in I.Q. and neuropsychological development.

Radionuclides are atoms that emit radiation and can be anthropogenic/man-maed. Radionuclides generally release their energy as alpha particles, beta particles or gamma rays. Radionuclides can be present in breast milk through ingestion by the nursing mother of contaminated food, air and/or water, or through the use of radio-pharmaceuticals. The effects on an infant can include, for example, increased cancer potential and thyroid problems.

In contrast with the filtration described in published U.S. Patent Application 2004/0178162A to Zucker-Franklin, entitled “Devices and Methods for Removal of Leukocytes from Breast Milk,” incorporated herein by reference, the present approach is directed to the removal of dissolved or suspended organic compositions or inorganic ions rather than size filtration to remove bacteria. The filtration media useful for the removal of leukocytes will not be effective for the removal of organic and inorganic toxins as described herein. However, different filtration media can be combined such that organic and/or inorganic toxins are removed as well as leukocytes.

In general, any breast shield design can be adapted for incorporation of a suitable filtration medium. However, it may be desirable to adjust the shape of the device to better provide for placement of the filtration medium without interfering with the placement of the device on the nursing mother. Similarly, the placement of the filtration medium can be selected to provide proper fit of the device.

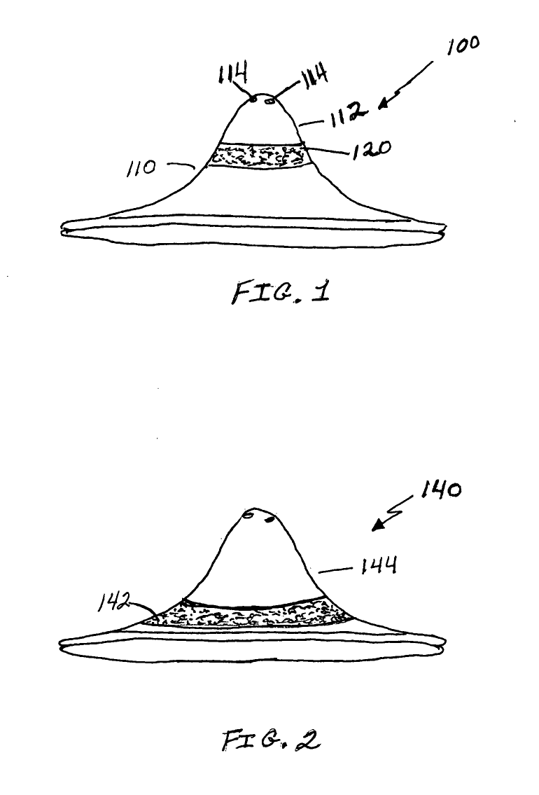

Referring to a representative embodiment in FIG. 1, a filtering nipple shield 100 comprises a nipple structure 110 with a tip 112. The tip has one or more holes 114 permitting passage of breast milk such that an infant may intake the breast milk. The filtering nipple shield 100 also comprises filter media 120 interior to the nipple shield 100 such that breast milk flows through the filter media 120 on its way to the holes 114. Nipple structure 110 can be shaped to conform to a mammalian female areola and nipple. As the mammalian breast can vary in shape and size, nipple shield 100 can take a variety of forms to accommodate these variations in breast size and shape. Filtering nipple shield 100, when placed over the areola and nipple of a mammalian breast, is shaped such that when it is sucked on by an infant, suction is created between the nipple shield 100 and surface of the mammalian breast. Commercial nipple shields are commercially available from companies such as Medela Inc. and Ameda. Commercial nipple shields can be adapted as nipple structures 110.

Filtering nipple shield 100 is generally made of flexible material such that nipple structure 110 can conform somewhat to the mammalian breast shape and size. Suitable materials for the nipple structure 110 can include, for example, rubber, latex, silicon, or the like, or combinations thereof.

Referring to FIG. 2, filtering nipple shield 140 comprises a filtering medium 142 at an alternative location in relationship with nipple structure 144. The filter media within the nipple shield can comprise a material that is capable of filtering-out endocrine disruptors such as polybrominated diphenyl ethers, polychlorinated biphenyls, dioxins, dibenzofurans, perchlorates, phthalates, and/or heavy metals and radionuclides. Suitable filtration media for the removal of organic compounds include, for example, activated carbon. The activated carbon can be within a porous block material with a polymer binder, such as described in U.S. Pat. No. 4,753,728 to VanderBilt et al., entitled “Water Filter,” incorporated herein by reference. However, the pressure drop across such a block structure can lead to undesirable nursing difficulties. Thus, it may be more desirable to place a granular activated carbon material within a porous structure that prevents the migration of the activated carbon while providing flow through the porous structure. Food grade activated carbon suitable for these applications is sold commercially by Calgon (Filtrasorb®) and U.S. Filter (AquaCarb® and BevCarb®).

Activated carbon filters can be effective in removing organic contaminants and endocrine disruptors such as halogenated hydrocarbons including PCB's and PBDE's, dioxins, dibenzofurans, and perchlorates, phthalates, and some heavy metals such as arsenic complexes, chromium complexes, and mercury complexes. The activated carbon filter material can be hydrophobic or hydrophilic, and can be granular with a mesh size selected to avoid migration of the activated carbon while providing a suitable surface area to remove desired contaminants. The addition of cationic and anionic resins that sorb cations and anions assists in filtering radionuclides and heavy metals from the breast milk. For example, radium can be removed by including sorbents, for example, acrylic fibers or resins impregnated with manganese dioxide, and non-sodium cation exchangers such as hydrogen ions and calcium ions. Carbion™ ion exchanger, available from Lenntech, for example, can be used as an ion exchanger to remove heavy metals.

The filter media can be contained within a porous membrane that allows for relatively easy flow of breast milk through the filter material and filter media. The filter material can be used to keep the filter media localized and contained in a disc or packet, or held within a porous silicon or porous rubber structure. Suitable materials for the filter material include, for example, a woven material, such as polyester or other woven polymer or a nonwoven material, such as a porous plastic material. The porosity can be chosen to keep the granular filtration medium within the membrane while providing for suitable milk flow. The membrane with the filtration medium can be molded into the nipple shield, attached within the nipple shield through welding, adhesive bonding or the like, or wedged releasably within the nipple shield with friction. The nipple shield can be discarded after each use, or cleaned and/or sterilized for reuse.

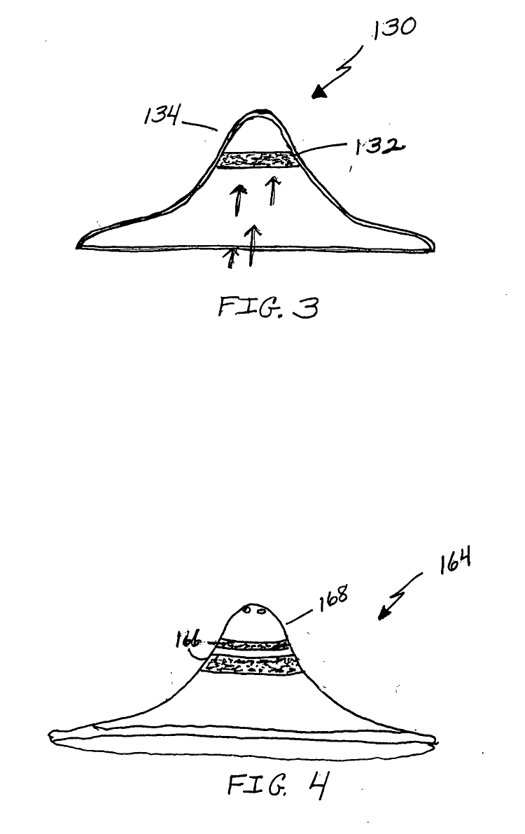

Referring to FIG. 3, filtering nipple shield 130 comprises filtering media 132, in this embodiment, positioned in the tip 134 of the filtering nipple shield 130. The filtering nipple shield 130 is shown in a cross-sectional view such that the directional flow of breast milk through the filter media 132 is shown by the positioning of the arrows. Referring to FIG. 4, there is shown another embodiment of a filtering nipple shield 164, wherein a plurality of filtering media 166 is placed within the filtering nipple shield tip 168. The plurality of filtering media 166 is placed sequentially within the nipple tip 168, such that the breast milk passes through a plurality of filters prior to being ingested by a feeding infant.

In general, the filter element can be permanently or releasably connected to the remaining portions of the nipple shield. Permanent connections can be formed with molding or adhesives or the like. Releasable connections can be formed with friction elements such that the filter remains in position during use but can be pulled out when desired. Thus, if the filter has a significantly longer or shorter life time than the other portions of the nipple shield, the elements can be independently replaced if the filter element is releasably attached.

Filtering nipple shield embodiments, such as these described above, provide for direct filtering of breast milk as the milk is ingested by a suckling infant. Alternatively, the milk can be collected for subsequent ingestion by an infant. In these embodiments, the breast milk can be filtered during the collection process or at the point of ingestion. For example, a filter can be attached to a bottle that holds that breast milk for ingestion. These filtering bottles similarly can be used to filter other liquids, such as cow's milk, sheep's milk, juices or the like prior to ingestion. In general, the filter medium can be placed along the flow path from the storage portion of the bottle to the bottle tip from which the liquid is consumed. A representative embodiment is presented in the following.

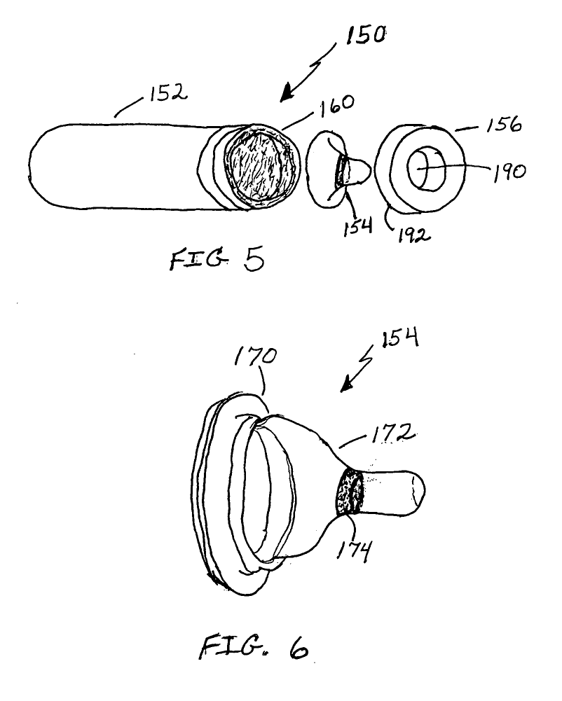

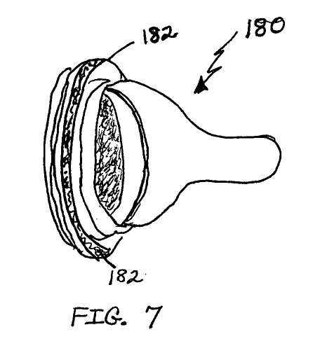

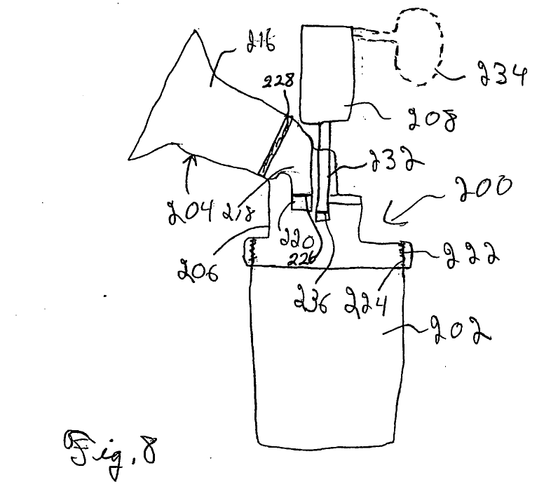

Referring to FIG. 5, a filtering bottle 150 comprises a storage compartment 152, a bottle nipple 154 and a cap 156. Breast milk that has been pumped and saved for future use or another liquid can be poured into filtering bottle 150 after sterilization. Storage compartment 152 can have conventional dimensions for easy holding and for storage of an appropriate quantity of liquid. Storage compartment 152 can comprise a disposable bag or the like to hold the liquid rather than directly placing the liquid into the storage compartment. Storage compartment 152 has an attachment portion 160 for the attachment of cap 156. Attachment portion 160 can comprise threads or the like for the attachment of embodiments in which cap 156 comprises mated threads. Alternatively, a clamp or the like can be used to secure cap 156 with attachment portion 160 in which cap 156 and attachment portion 160 have suitable flanges to engage the clamp. Similarly, any other suitable attachment structure can be used. FIGS. 6 and 7 show alternate bottle nipples 154, 180 that can be used with a storage compartment 152.

Referring to FIG. 6, bottle nipple 154 comprises a lip section 170, nipple portion 172 extending from lip section 170 and filter portion 174 within nipple portion 172. Lip section 170 has suitable dimensions for interfacing with attachment portion 160 and the positioning between attachment portion 160 and cap 156 such that bottle nipple can be held in place. Filter portion 174 can have similar structure and filter compositions as filtering medium 142 in the breast shield, as described above. However, the placement of the filter medium can be positioned without regard for interference with the placement of the nipple portion over the nursing mother's breast since bottle nipple 154 is just placed on a bottle. Thus, referring to FIG. 7, bottle nipple 180 comprises a filter element 182 placed across the mouth of bottle nipple 180, as an alternative or in addition to the placement of the filter element further toward the tip of the bottle nipple. As with the filtering nipple shield, bottle nipple 180 can comprise a plurality of filter elements. Also, the filter element can be secured across the mouth of the storage compartment without direct attachment with the bottle nipple.

Cap 156 comprises an orifice 190 and a cap attachment section 192. The bottle nipple 154, 180 fits through orifice 190 for attachment to storage compartment 152. Cap attachment section 192 can comprise threads mated for engaging attachment portion 160 or other suitable structure for engaging attachment portion 160 directly or with a clamp or the like. In alternative embodiments, the filtering baby bottle may not include a cap element. For example, the lip section of the nipple portion can have an elastic seal that extends over and releasably grips the attachment portion of the storage compartment.

For use, the storage compartment 152 and cap 156 are attached such that lip 170 or a separate gasket or the like provides a seal so that liquid does not leak out of the bottle 150. When the infant sucks on bottle nipple 154 to obtain milk from filtering bottle 150, the milk passes through filtering portion 174. Upon emptying the bottle, bottle nipple 154 and/or filter portion 174 can be removed and discarded. Alternatively, bottle nipple 154 and/or filter portion 174 can be cleaned, sterilized and reused. In further embodiments, new or sterilized filter portion 174 can be placed in the interior of bottle nipple 154. Storage compartments 152 can be formed of suitable plastics. Bottle nipple 154 and filter portion 174 can generally be made of similar corresponding materials described above with respect to the nipple shield.

In a further embodiment, a filter is placed as an integral part of a breast milk pumping device. The breast milk pumping device generally can comprise any type of filtration medium to filter the breast milk. In some embodiments, the filtration medium in the breast milk pump comprises activated carbon and/or an ion sorptive medium, such as an ion exchange resin. The activated carbon filter material can be granular with a mesh size in the range of from 0.025 mm to 4.75 mm in width. The filter media 40 can be contained in a filter packet, where the covering filter material allows for passage of the filtered breast milk. The packet material can be comprised of nonwoven and/or woven material. Breast milk pumps are available from manufactures, such a Medela Inc. and Ameda. Commercial designs can be adapted for filtration or new designs can be used.

In general, a filtering breast milk pump comprises a collection reservoir, a collection cup, a filter in the flow path from the collection cup to the collection reservoir and a pump. The collection reservoir can be any suitable size and shape. The collection cup generally is designed to fit reasonably and comfortably over a female mammalian breast for collecting the milk. The cup generally has a neck extending from the cup that leads to a channel directed to the reservoir. The filter is positioned within the flow path from the woman's breast to the reservoir. Thus, the filter can be placed, for example, in the neck of the cup or in the channel leading to the reservoir.

The pump can be connected to the remaining portions of the device in a range of configurations. Many configurations have been described. The pump can be a manual pump in which the user pumps the device to provide the desired degree of pressure differential. Manual pumps generally can have a handle connected to a baffle, an elastic bladder or the like to perform the pumping action. Alternatively or additionally, a motorized pump can be used. A motorized pump has the advantage that a person does not have to provide the pumping action. An example of a breast pump construction that can be adapted for manual or automatic suction pumps is described further in U.S. Pat. No. 4,759,747 to Aida et al., entitled “Breast Pump Including Pressure Adjusting Means,” incorporated herein by reference. Another representative breast pump design is discussed in U.S. Pat. No. 6,110,141 to Nüesch, entitled “Breast Pump and Overflow Protection for an Apparatus for Sucking a Body Fluid Off,” incorporated herein by reference. The present filtration designs for filtering milk prior to entering the reservoir are in stark contrast with designs intended to prevent fouling of the pump, which generally are designed to prevent passage of milk rather than filtering the milk.

A schematic view of a representative embodiment of a filtering breast milk pump is shown in FIG. 8. Breast pump 200 comprises a reservoir 202, a funnel shaped cup 204, a manifold 206 and a pump 208. Reservoir 202 holds the filtered breast milk. The reservoir can be accessed for the removal of the filtered milk and for subsequent cleaning of the reservoir, if desired. Cup 204 is designed to fit over the breast of the nursing mother. Cup 204 has a funnel shaped cone 216 that tapers into neck 218. Neck 218 transitions into conduit 220 leading to reservoir 202 or similarly is fluidly connected to such a conduit. Milk flowing through the neck is collected in the reservoir.

Manifold 206 provides for connections between reservoir 202, cup 204 and pump 208. Manifold 206 can have a connection 222 such as screw elements for the removal of reservoir 202 from manifold 206, in which case reservoir 202 comprises mated screw threads 224. It can be advantageous to provide a screw lid to close the reservoir to obviate the need to transfer the milk to a separate storage container. In other embodiments, manifold 206 can be fixed to a reservoir with a resealable opening to provide access for the removal of the filtered milk.

A filter element generally is located within the flow from cup 204 to reservoir 202. As shown in FIG. 8 a filter element 228 is shown in cup 204, and filter element 226 is shown in conduit 220. Pump 200 can include one or both representative filter elements 228, 226 or other filter elements along the flow pathway. Generally filter elements 228, 226 can be removed for cleaning and/or replacement. Filter elements 228, 226 can be formed from similar materials and similar filtration media as filter elements described with respect to FIGS. 1-7.

Pump 208 is fluidly connected to pump conduit 232 that provided for creating negative pressure within reservoir 202. Pump 208 can be a motorized pump or a manual pump. An optional manual squeeze ball 234 is shown in phantom lines in FIG. 8. An optional air filter 236 is shown within pump conduit 232 to keep milk from entering the pump. Pumping is performed as needed, and the filter elements generally do not significantly alter the pumping process.

Generally, the devices described herein as well as potentially other devices can be used to practice a method of removing organic toxins and/or inorganic toxins, such as halogenated endocrine disruptors, phthalates, radionuclides and heavy metals from breast milk. As noted above, the method can be used for the direct filtration during the suckling process of an infant or for the filtration of stored milk at collection, at delivery or during some subsequent period between collection and delivery. In some embodiment, the filter comprises a filtration medium with activated carbon, since activated carbon is effective at the removal of halogenated organic compounds. However, other suitable filtration media can be used.

In one embodiment, a filtering nipple shield 100 can be placed over an areola and nipple region of a female mammalian breast. The infant would suck on the nipple thereby creating suction between the nipple shield and the mammalian breast such that enough suction is created to cause milk to flow from the mammalian nipple through the filtering nipple shield. The breast milk flows through the filter media prior to exiting the nipple shield.

In another embodiment, a filtering baby bottle can be used to filter toxins from breast milk. An infant can suck on the nipple of the baby bottle to obtain filtered breast milk from the interior of the bottle. The breast milk in the baby bottle passes through the filter, pulled by the sucking action of the infant.

Alternatively, a filtering breast milk pump can be used as a method of removing toxins from breast milk. In particular, the breast milk pump filter is placed within the breast milk pump mechanism and filters the breast milk as it is pumped from the breast into a collection receptacle. The filtered breast milk collected can then be fed to an infant in a baby bottle. Alternatively, the collected breast milk could be filtered again by placing the breast milk in a baby bottle configured with a baby bottle filter, as described above.

The embodiments described above are intended to be illustrative and not limiting. Additional embodiments are within the claims. Although the present invention has been described with reference to preferred embodiments, workers skilled in the art will recognize that changes may be made in form and detail without departing from the spirit and scope of the invention. Any incorporation by reference of documents above is limited so that no subject matter is incorporated that is contrary to the explicit disclosure herein.

1. A filtering nipple shield comprising:
a nipple structure comprising a nipple shaped material having an interior and a tip with one or more holes; and
a first filter comprising a first filtration medium, the filter being positioned in the interior of the nipple structure wherein the filtration medium effectively removes organic toxins or inorganic toxins from a liquid contacting the filtration medium.

2. The filtering nipple shield of claim 1 wherein the nipple shield comprises material selected from the group consisting of latex, silicon and rubber.

3. The filtering nipple shield of claim 1 wherein the filter comprises activated carbon.

4. The filtering nipple shield of claim 1 wherein the filter comprises a resin that sorbs ions.

5. The filtering nipple shield of claim 1 wherein the first filter comprises a second filtration media.

6. The filtering nipple shield of claim 1 wherein the first filtration medium is granular and wherein the filter further comprises a porous cover over the first filtration media.

7. The filtering nipple shield of claim 1 further comprising a second filter comprising a second filtration medium.

8. The filtering nipple shield of claim 7 wherein the first filtration medium comprises activated carbon and the second filtration medium comprises an ion exchange resin.

9. A baby bottle comprising:
a container comprising an opening and an attachment portion;
a nipple comprising a lip with a flange, a protrusion and a tip with one or more holes wherein the nipple fits over the opening of the container; and
a filter comprising a filtration medium that effectively removes organic toxins or inorganic toxins from liquid contacting the filtration medium, the filter being positioned within the bottle between the container and the tip of the nipple.

10. The baby bottle of claim 9 wherein the filter is secured within the protrusion of the nipple.

11. The baby bottle of claim 9 wherein the filter extends across the opening of the container.

12. The baby bottle of claim 9 wherein the filter comprises a granular filtration medium within a porous cover.

13. The baby bottle of claim 9 wherein the filtration medium comprises activated carbon.

14. The baby bottle of claim 9 wherein the filtration medium comprises an ion exchange resin.

15. The baby bottle of claim 9 wherein the lip of the nipple comprises a seal that elastically stretches over the attachment portion of the container to secure the nipple to the container.

16. The baby bottle of claim 9 further comprising a cap comprising an aperture and a cap attachment portion wherein the tip of the nipple fits through the aperture and the cap attachment portion engages the attachment portion of the container to secure the flange of the nipple between the container opening and the cap.

17. A filtering breast pump comprising:
a reservoir;
a breast cup comprising a shaped cone and a neck extending from the cone fluidly connected to the reservoir such that breast milk generated in the breast cup flows through the neck into the reservoir;
a filter within the flow path between the breast cup and the reservoir; and
a pump positioned to evacuate the reservoir to induce pumping through the neck to the breast cup.

18. The filtering breast pump of claim 17 wherein the filter is placed within the neck of the breast cup.

19. The filtering breast pump of claim 17 wherein the filter is at the opening of the neck into the reservoir.

20. The filtering breast pump of claim 17 wherein the filtration medium comprises activated carbon.

21. The filtering breast pump of claim 17 wherein the filtration medium effectively removed organic toxins or inorganic toxins from liquid contacting the filtration medium.

22. The filtration breast pump of claim 17 wherein the pump comprises a manual pump.

23. The filtration breast pump of claim 17 wherein the pump comprises an electric pump.

24. A method for removing organic toxins or inorganic toxins from breast milk, the method comprising filtering breast milk with a filter effective to remove organic toxins or inorganic toxins.

25. The method of claim 24 wherein the filtering of the breast milk is performed with a nipple shield comprising a filter.

26. The method of claim 24 wherein the filtering of the breast milk is performed using a baby bottle comprising a container, a nipple secured to the container and filter within the bottle to filter the contents from the container prior to delivery through the nipple.

27. The method of claim 24 wherein the filtering of the breast milk is performed using a breast pump that filters milk collected within a breast pump prior to delivery into a reservoir.
